(19) United States
(12) Patent Application Publication
(10) Pub. No.: US 2006/0121136 A1
(43) Pub. Date: Jun. 8, 2006

(54) Hypotensive agent and method for producing same

(75) Inventors: Syuichi Oka, Ibaraki (JP); Akinori Tsuruda, Ibaraki (JP); Yasuhiro Kawano, Ibaraki (JP); Susumu Maruyama, Ibaraki (JP); Yoshimitsu Yamazaki, Ibaraki (JP); Miyuki Kikuchi, Tokyo (JP); Hideaki Ono, Tokyo (JP)

Correspondence Address: BROWDY AND NEIMARK, P.L.L.C.;624 NINTH STREET, NW, SUITE 300, WASHINGTON, DC, 20001-5303, US

(73) Assignee: National Institute of Advanced Industrial Science and Technology, Tokyo (JP)

(21) Appl. No.: 10/522,678

(22) PCT Filed: Oct. 28, 2004
(86) PCT No.: PCT/JP04/16369

§ 371 Date: Jan. 19, 2006

(30) Foreign Application Priority Data
Nov. 18, 2003 (JP) 2003387404

(51) Int. Cl. A61K 36/48; A61K 31/715; C08B 37/00

(52) U.S. Cl. 424/757; 514/54; 536/123

(57) ABSTRACT

Disclosed is a hypotensive agent that is safe and inexpensive. The agent contains an oligosaccharide that comprises at least one type of hexose and has at least one 1-6 bond.

The present application is a continuation of PCT/JP2004/16369 filed on Oct. 28, 2004 and claims priority under 35 U.S.C. §119 of Japanese Patent Application No. 387404/2003 filed on Nov. 18, 2002.

### TECHNICAL FIELD

The present invention relates to a hypotensive agent that contains an oligosaccharide having a specific structure, and to a method for producing the agent.

### BACKGROUND ART

Since early times, hypertension has been a problem. Hypertension causes cerebral hemorrhage disease, cardiopathy, kidney trouble, etc., and it is important to prevent hypertension and to inhibit the progress of hypertension. Especially for aged persons, hypertension may bring about serious problems in many cases. At present, in addition, hypertension is a lifestyle disease and is problematic not only for aged persons but also for younger persons.

JP-A2002-272420 discloses an anti-hypertension-related ingesta composition that contains sodium alginate oligosaccharide as the active ingredient thereof.

The hypotensive agent that is utilized in the above-mentioned blood pressure-lowering foods and medicines requires a plurality of materials, and many of the materials are expensive. In addition, when taken habitually, some of them may cause a problem in point of safety, etc. However, hypertension is now a general problem with all men of today, and inexpensive and safe hypotensive agents are much desired. Further, aged persons who take different kinds of medicines as combined and men of today who may take many nutrient-supplementary foods such as supplements desire a hypotensive agent which they may take along with these with no or little harm to them.

### DISCLOSURE OF THE INVENTION

We, the present inventors have investigated to find out a hypotensive agent that solves the above-mentioned problems. Accordingly, the invention is to provide a hypotensive agent that is safe and inexpensive.

Given the situation as above, we have assiduously studied and, as a result, have found that an oligosaccharide having a specific structure has a blood pressure-lowering effect. In particular, we have investigated in detail the bonding formation among monosaccharides, and have found that the effect significantly varies depending on the bonding formation. Concretely, the invention takes the following means.

(1) A hypotensive agent containing an oligosaccharide that comprises at least one type of hexose and has at least one 1-6 bond.

(2) The hypotensive agent of (1), wherein the hexose is selected from the group consisting of glucose, galactose, mannose, altrose, talose, allose, idose, gulose, fructose, psicose, tagatose, sorbose, mannitol, altritol, talitol, iditol, galactitol, allitol, gulitol and glucitol.

(3) The hypotensive agent of (1), wherein the hexose is selected from the group consisting of glucose, galactose, mannose and fructose.

(4) The hypotensive agent of (1), wherein the hexose is selected from the group consisting of glucose, galactose and fructose.

(5) The hypotensive agent of any of (1) to (4), wherein the oligosaccharide contains constitutive unit of Glc (α1-6) Glc, Glc(β1-6)Glc, Gal (α1-6)Glc, Gal(β1-6)Gal and/or Glc (α1-6)Fru.

(6) A hypotensive agent containing an oligosaccharide that consists of glucose and/or galactose and has at least one 1-6 bond.

(7) The hypotensive agent of any of (1) to (6), wherein the oligosaccharide is that all hexoses bond by 1-6 bound.

(8) The hypotensive agent of any of (1) to (7), wherein the oligosaccharide is biose and/or triose.

(9) The hypotensive agent of any of (1) to (8), wherein the oligosaccharide is derived from leguminous plants.

(10) The hypotensive agent of any of (1) to (8), wherein the oligosaccharide is derived from black soybeans.

(11) The hypotensive agent of any of (1) to (10), wherein the oligosaccharide does not contain constitutive unit of Glc(α1-3)Fru and Glc(α1-2B)Fru.

(12) The hypotensive agent of any of (1) to (11), wherein the hexose is not raffinose.

(13) A method for producing the hypotensive agent of any of (1) to (12), which comprises extracting a leguminous plant with water or an organic solvent.

(14) A method for producing the hypotensive agent of any of (1) to (12), which comprises extracting a leguminous plant with water or an organic solvent, and then concentrating the resulting extract.

(15) A method for producing the hypotensive agent of any of (1) to (12), which comprises extracting a black soybean with water or an organic solvent.

### ADVANTAGE OF THE INVENTION

The invention employs an oligosaccharide that consists of a hexose and has a 1-6 bond, and it provides a safe, readily-producible and low-cost hypotensive agent.

### BRIEF DESCRIPTION OF THE DRAWING

FIG. 1 shows ACE inhibiting activity of various oligosaccharides.

### BEST MODE FOR CARRYING OUT THE INVENTION

The invention is described in detail hereinunder. In this description, the numerical range expressed by the wording “a number to another number” means the range that falls between the former number indicating the lowermost limit of the range and the latter number indicating the uppermost limit thereof.

The oligosaccharide for use in the invention comprises at least one type of hexose and has at least one 1-6 bond. The hexose for use in the invention includes, for example, glucose, galactose, mannose, altrose, talose, allose, idose, gulose, fructose, psicose, tagatose, sorbose, mannitol, altritol, talitol, iditol, galactitol, allitol, gulitol and glucitol. Preferably, it is not raffinose. More concretely, the oligosaccharide for use in the invention consists of hexoses which is at least one hexose selected from the group consisting of glucose, galactose, mannose and fructose, and has at least one 1-6 bond. Preferably, the oligosaccharide contains constitutive unit of Glc (α1-6)Glc, Glc(β1-6)Glc, Gal (α1-6)Glc, Gal(β1-6)Gal and/or Glc (α1-6)Fru. More preferably, the oligosaccharide comprises only glucose and/or galactose and has at least one 1-6 bond, or contains a constitutive unit of Glc(α1-6)Fru.

The oligosaccharide includes 2-20 monosaccharides, preferably from 2-10 monosaccharides, more preferably from 2-5 monosaccharides, even more preferably biose or triose. The 1-6 bond means that the 1-positioned carbon of one hexose bonds with the 6-positioned carbon of another hexose. The bonding may be in any form of α-bonding or β-bonding. The hexose for use in the invention may be any of D-form or L-form. The oligosaccharide for use in the invention contains at least one 1-6 bond, in which, however, it is desirable that all carbon-carbon bonds among all hexose are 1-6 bonds. When the oligosaccharide contains any other bond than 1-6 bond, then the other bond is preferably 1-2 bond and/or 1-4 bond.

The proportion of each hexose in the oligosaccharide is not specifically defined. For example, when the oligosaccharide comprises glucose, galactose, mannose and fructose, then their proportion is preferably such that the ratio of any one saccharide is 1 and the ratio of the other saccharide is from 0.1 to 10 each. In particular, when the oligosaccharide consists of glucose and galactose, then it is desirable that the ratio of galactose is 1 and the ratio of glucose is at most 0.5 or at most 0.4. When the oligosaccharide contains fructose, then it is desirable that at least one monosaccharide therein that constitutes 1-6 bond is fructose. In this case, it is more desirable that the monosaccharide that constitutes any other bond than 1-6 bond (e.g., 1-2 bond or 1-4 bond) is any other saccharide than fructose (preferably, glucose and/or galactose). Also preferably, the oligosaccharide of this case does not contain 1-3 bond. Needless to say, however, the oligosaccharide for use in the invention should not exclude any other oligosaccharide than those as above.

Further, the oligosaccharide for use in the invention preferably contains constitutive unit of Glc(α1-6)Glc, Glc(β1-6)Glc, Gal(α1-6)Glc, Gal(β1-6)Glc, Gal(α1-6)Gal, Gal(β1-6)Gal, Glc(α1-6)Man, Glc(α1-6)Fru. More preferably, it does not contain Glc(α1-3)Fru and Glc(α1-2β)Fru.

In the invention, for example, 1-6 bond at α-position may be represented by α1-6 bond; and β-bonding of 1-positioned carbon of glucose (Glc) and 6-positioned carbon of galactose (Gal) may be represented by Glc(β1-6)Gal.

One or more different types of oligosaccharides may be used in the invention. The oligosaccharides for use in the invention may be commercially-available products or may also be natural materials. We, the present inventors have widely investigated various natural materials existing in the natural world, and, as a result, have found that the above-mentioned oligosaccharides are contained in leguminous plants, especially in soybeans of leguminous plants, in particular, in soybeans, especially in black soybeans. There has heretofore been some suggestion indicating that oligosaccharides may be contained in such plants, but their content is minor and the type of the oligosaccharides to be in the plants is not specifically identified. This time, we, the present inventors have specifically identified the type of the oligosaccharides contained in black soybeans, and have clarified what components of those are effective for low blood pressure. In addition, we have further found that, when the plants are extracted with water or an organic solvent, then the intended components can be obtained.

The leguminous plants as referred to herein are meant to include not only soybeans but also leaves, roots, stems, etc. Preferably, leguminous plants are soybeans. Soybean may be natural ones, and may also widely contain any others such as dried or powdered ones. For example, industrial wastes such as broth of black soybeans (water obtained by extracting black soybeans) or curd refuse of soybeans may also be used in the invention. From the environmental viewpoint, their positive use is desired these days, and from this point of view, the invention is favorable.

In the invention, the leguminous plants are extracted with water or an organic solvent. Water is meant to include any of cold water, warm water, hot water and steam. Not specifically defined, the organic solvent for use in the invention may be any one capable of attaining the object of the invention, or that is, anyone having the ability to dissolve oligosaccharides. It is meant to indicate any one comprising water or any other organic solvent. Concretely, the solvent includes alcohols (e.g., methanol, ethanol), acetone, hexane, petroleum ether, petroleum benzene, ethyl acetate, methylene chloride, ether, isopropyl ether, chloroform. If desired, the extraction may be effected in two or more stages.

Concretely, a leguminous plant is dipped and extracted in the above-mentioned water or the like. The extraction time may be suitably determined depending on the type or the stored condition of the plant, and on the type of water or the organic solvent to be used. Preferably, it falls between 30 minutes and 1 week, more preferably between 1 and3 days. The extraction may be effected in a mode of static extraction or may be in a mode of shaking extraction. Preferably, the extraction is repeated a few times while exchanging the solvent for a new one.

The extraction condition including the time and the temperature and the pH of the extraction or elution bath may be suitably determined depending on the plant to be selected.

The hypotensive agent of the invention may be used for low blood pressure in humans and animals. For example, it may be used for medicines, pharmaceutical compositions or health foods.

When it is utilized for hypotensive agent or its active ingredient as medicines or quasi-drugs, the oligosaccharide of the invention may be directly administered, but is preferably administered as a pharmaceutical composition thereof that can be produced in any method well known to those skilled in the art. The pharmaceutical composition includes, for example, tablets, capsules, powders, fine granules, granules, liquids, and syrups. The pharmaceutical composition may be produced by adding pharmacologically and pharmaceutically acceptable additives to the active ingredient. Examples of the pharmacologically and pharmaceutically acceptable additives are, for example, vehicle, disintegrator or disintegration aid, binder, lubricant, coating agent, colorant, diluting agent, base, solubilizer or disintegration aid, isotonicating agent, pH controlling agent, stabilizer, propellant and adhesive agent. The pharmaceutical composition may contain one or more other hypotensive agents. The dose of the medicine of the invention is not specifically defined, and may be suitably determined depending on the type of the active ingredient to be in the medicine. Further, it may be increased or decreased in accordance with various factors that should be generally taken into consideration in using it, such as the body weight and the age of the patient, the type and the condition of the disease, and the administration route for the medicine. In general, the dose/adult/day may fall between 0.000001 g and 100 g, preferably between 0.004 g and 40 g. The administration route for the medicine is not also specifically defined. For example, the medicine may be administered intravenously as injection or transfusion, or orally.

The health foods include dietary foods for hypertensives and those for prevention of hypertension. They may also be usable for health-supplementary foods, nutrient-supplementary foods, supplements, drinks, seasonings, retorted foods, everyday dishes and others for hypertensives and those for prevention of hypertension.

### EXAMPLES

Examples of the invention are described below. The materials, and their amount, proportion, treatment condition and treatment order described in the examples may be suitably varied without overstepping the sprit and the scope of the invention. Accordingly, the invention should not be limitatively interpreted by the following examples. (Angiotensin-converting enzyme inhibiting activity of oligosaccharide)

The blood pressure-lowering activity of the oligosaccharide for use in the invention was investigated by determining the angiotensin-converting enzyme inhibiting activity (hereinafter referred to as ACE inhibiting activity) thereof. Concretely, various oligosaccharides mentioned below were analyzed for their ACE inhibiting activity.
1) Glc(α1-1α)Glc: α,α-trehalose (sold by Wako Jun-yaku, Lot No. KP7915),
2) Glc(α1-1β)Glc: α,β-trehalose (sold by Sigma, Lot No. T-0299),
3) Glc(β1-1β)Glc: β,β-trehalose (sold by Sigma, Lot No. T-0917),
4) Glc(α1-2)Glc: kojibiose (sold by Sigma, Lot No. T-0299),
5) Glc(β1-2)Glc: sophorose (sold by Sigma, Lot No. S-1404),
6) Glc(α1-3)Glc: nigerose (sold by Wako Jun-yaku, Lot No. 142-06433),
7) Glc(β1-3)Glc: laminaribiose (sold by Sigma, Lot No. L-6384),
8) Glc(α1-4)Glc: maltose (sold by Sigma, Lot No. M-9171),
9) Glc(β1-4)Glc: cellobiose (sold by Nacalai, Lot No. 075-11),
10) Glc(α1-6)Glc: isomaltose (sold by Sigma, Lot No. I-7253),
11) Glc(β1-6)Glc: gentiobiose (sold by Sigma, Lot No. G-3000),
12) Gal(α1-6)Glc: melibiose (sold by Sigma, Lot No. M-5500),
13) Gal(β1-6)Gal (sold by Sigma, Lot No. G-5643),
14) Glc(α1-6) glucitol:Glc(α1-6) mannitol (=50:50) (sold by Sigma, Lot No. P8583-500),
15) Glc(α1-6)Fru: palatinose (sold by Sigma, Lot No. M-5500),
16) Fru(β2-1α)Glc: sucrose (sold by Wako Jun-yaku, Lot No. 192-00012), and
17) Glc(α1-3) Fru(β2-1α) Glc: melezitose (sold by Nacalai, Lot No. 214-01).
   In these, Glu means glucose; Gal means galactose; and Fru means fructose.

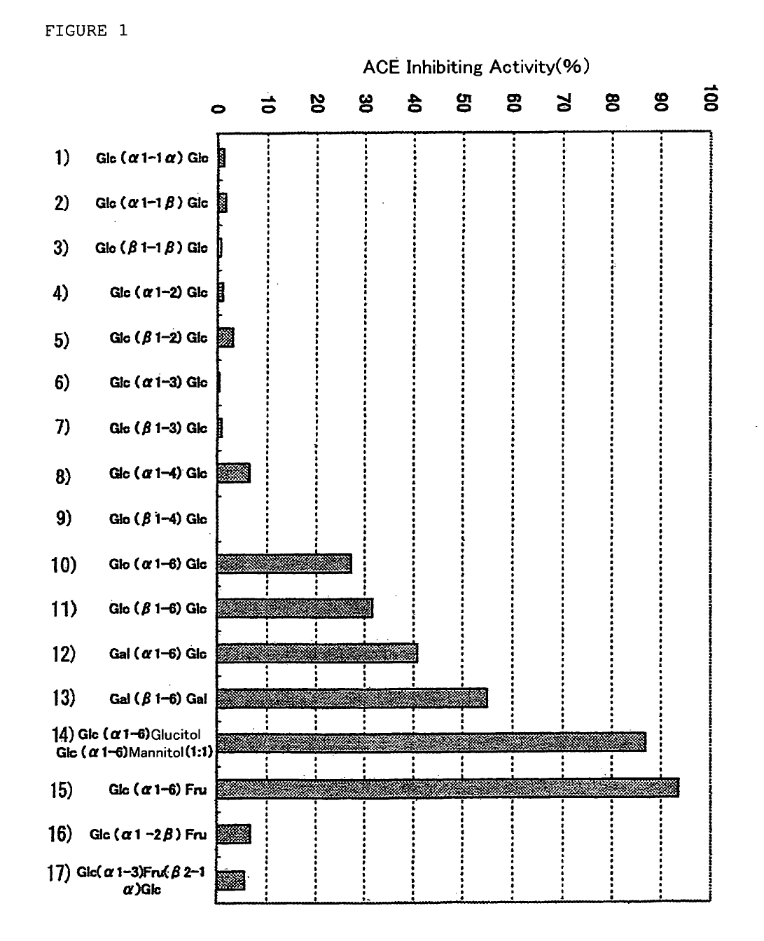

Each oligosaccharide as above was dissolved in water (MilliQ water). Because of the solubility thereof, the sample was dissolved to have a final concentration of 10, 30 or 100 mM at the time when its activity is determined (Table 1). Next, with cooling with ice, 30 μl of the sample (Nos. 1 to 17 in Table 1) was put into a test tube. Then, 100 μl of ACE (sold by Wako Jun-yaku, Lot No. A6778) in a borate buffer (60 mU/ml) (pH 8.3) was added to each sample. As a blank, one also added with 250 μl of 1 N HCl serving as a reaction stopper was prepared. These were pre-incubated at 37° C. for 5 minutes. Further, 250 μl of a borate buffer (pH 8.3) containing as a substrate, 7.6 mM Hip-His-Leu (sold by Sigma, Lot No. 012-02195) and 608 mM sodium chloride (sold by Wako Jun-yaku, Lot No. 191-01665) was added. These were reacted at 37° C. for 30 minutes. Then, 250 μl of 1 NHCl was added and stirred, and the reaction was stopped. Then, 1.5 ml of ethyl acetate (sold by Wako Jun-yaku, Lot No. 051-00356) was added, and the thus-liberated hippuric acid was extracted out. After thus extracted, it was centrifuged at 3000 rpm for 10 minutes, and 0.5 ml of the supernatant (ethyl acetate layer)was recovered. The thus-recovered ethyl acetate was removed under reduced pressure, and 4 ml of water was added and stirred. The absorption of the dissolved hippuric acid at 228 nm (OD) was measured. The borate buffer used herein was prepared from Na₂B₄O₇ (sold by Wako Jun-yaku, Lot No. 194-01415) and H₃BO₃ (sold by Wako Jun-yaku, Lot No. 012-02195) so that its pH could be 8.3. The data obtained were computed according to the calculation formula mentioned below, and the ACE inhibiting activity (%) of each sample was obtained as in Table 1 and FIG. 1.
ACE Inhibiting Activity (%)={[(solvent OD−solvent blank OD)−(sample OD−sample blank OD)]/(solvent OD−solvent blank OD)}×100.

TABLE 1

| Sample | Concentration | ACE Inhibiting | |
|---|---|---|---|
| No. | (mM) | Activity (%) | |
| 1 | 30 | 1.2 | Comparison |
| 2 | 30 | 1.6 | Comparison |
| 3 | 30 | 0.6 | Comparison |
| 4 | 10 | 1.0 | Comparison |
| 5 | 30 | 3.0 | Comparison |
| 6 | 10 | 0.4 | Comparison |
| 7 | 30 | 0.8 | Comparison |
| 8 | 100 | 6.3 | Comparison |
| 9 | 30 | −1.0 | Comparison |
| 10 | 100 | 27.2 | The invention |
| 11 | 100 | 31.5 | The invention |
| 12 | 100 | 40.7 | The invention |
| 13 | 100 | 54.9 | The invention |
| 14 | 100 | 86.8 | The invention |
| 15 | 100 | 93.5 | The invention |
| 16 | 30 | 6.6 | Comparison |
| 17 | 30 | 5.5 | Comparison |

As in Table 1 and FIG. 1, samples Nos. 10 to 15 had a high ACE inhibiting activity. Comparing sample No. 10 (Glc(α1-6)Glc) with sample No. 11 (Glc(β1-6)Glc) showed no difference in the ACE inhibiting activity between the isomers. FIG. 1 is a graph of the data in Table 1.

(Extraction of Blood Pressure-Lowering oligosaccharides from Black Soybeans)

4 liters of water was added to 1 kg of black soybeans, and heated at 10020 C. for 10 to 50 minutes to obtain a broth of black soybeans. Next, DEAE Toyopearl anion-exchange resin was added to the broth (12 liters), and left overnight at room temperature. Then, the supernatant was recovered through filtration. CM Toyopearl cation-exchange resin (sold by toyoboo) was added to the thus-recovered supernatant, and left overnight at room temperature. The component adsorbed by the CM Toyopearl cation-exchange resin was eluted out into MilliQ water. The resulting fraction was analyzed for its ACE inhibiting activity in the same manner as in Example 1, and this confirmed that the fraction had the intended activity.

The fraction thus obtained herein was freeze-dried, and the resulting residue was again dissolved in MilliQ water to prepare a 20-fold concentrated solution. The concentrated solution was applied to a C18 column, in which the adsorbed component was eluted out into MilliQ water and the active fraction was thus obtained. The active fraction was analyzed through chromatography with LH-20 column (two times).

The active fraction was analyzed through 1H-NMR and 13C-NMR. The 13C-NMR gave a spectrum peculiar to saccharide (anomeric carbon: at around 100 ppm). Further, the data of the sample were compared with the 1H-NMR and 13C-NMR spectral data of mono saccharide (standard) and Gal (α1-6)Glc, melibiose (standard), and analyzed in detail. As a result, it was confirmed that the active ingredients of the sample were two oligosaccharides of Gal(α1-6)Glc and Gal(α1-6)Gal(α1-6)Glc.

TABLE 2

| Type of Saccharide | | | | Melibiose | Active Fraction of Broth of Black Bean | |
|---|---|---|---|---|---|---|
| Monosaccharide | | ¹³C-NMR (ppm) | | ¹³C-NMR (ppm) | ¹³C-NMR (ppm) | ¹H-NMR (ppm) |
| (standard) | | Document | Found | Measured value | Measured value | Measured value |
| 1-O-Me-α-Gal | C₁ | 100.50 | 100.06 | 99.32 | 98.95 | 4.87 (d, 2 Hz) |
| | C₂ | 69.40 | 68.85 | 69.57 | 69.24 | 3.70-3.72 |
| | C₃ | 70.60 | 70.14 | 70.32 | 70.43 | 3.70-3.72 |
| | C₄ | 70.40 | 69.89 | 70.32 | 70.43 | 3.72-3.78 |
| | C₅ | 71.80 | 71.34 | 71.21 | 71.91 | 3.86-3.90 |
| | C₆ | 62.30 | 61.90 | 62.23 | 62.10 | 3.62 |
| 1-O-Me-α-Gal | C₁ | 100.50 | 100.06 | — | 98.80 | 4.87 |
| | C₂ | 69.40 | 68.85 | — | 69.40 | 3.66-3.70 |
| | C₃ | 70.60 | 70.14 | — | 70.19 | 3.82-3.85 |
| | C₄ | 70.40 | 69.89 | — | 69.61 | 4.06-4.11 |
| | C₅ | 71.80 | 71.34 | — | 69.78 | 4.00-4.07 |
| | C₆ | 62.30 | 61.90 | — | 67.40 | 3.52-3.56, 3.76-3.80 |
| β-Glc | C₁ | 96.50 | 96.98 | 97.19 | 97.07 | 4.54 (d, 8 Hz) |
| | C₂ | 74.80 | 75.21 | 75.16 | 75.02 | 3.13 (dd, 8, 8 Hz) |
| | C₃ | 76.40 | 76.84 | 75.47 | 76.97 | 3.37 |
| | C₄ | 70.30 | 70.68 | 70.60 | 69.78 | 4.00-4.07 |
| | C₅ | 76.60 | 77.00 | 77.00 | 75.11 | 3.51 |
| | C₆ | 61.50 | 61.85 | 67.03 | 67.40 | 3.52-3.56, 3.76-3.80 |
| α-Glc | C₁ | 92.70 | 93.17 | 93.30 | 93.18 | 5.11 (d, 2 Hz) |
| | C₂ | 72.14 | 72.56 | 72.54 | 70.62 | |
| | C₃ | 73.40 | 73.85 | 74.06 | | |
| | C₄ | 70.40 | 70.73 | 70.69 | 70.83 | |
| | C₅ | 72.10 | 72.50 | 72.07 | 72.40 | |
| | C₆ | 61.30 | 61.69 | 66.94 | 67.30 | 3.52-3.56, 3.76-3.80 |

The present disclosure relates to the subject matter contained in Japanese Patent Application No. 387404/2003 filed on Nov. 18, 2002 which is expressly incorporated herein by reference in its entirety.

The foregoing description of preferred embodiments of the invention has been presented for purposes of illustration and description, and is not intended to be exhaustive or to limit the invention to the precise form disclosed. The description was selected to best explain the principles of the invention and their practical application to enable others skilled in the art to best utilize the invention in various embodiments and various modifications as are suited to the particular use contemplated. It is intended that the scope of the invention not be limited by the specification, but be defined claims set forth below.

1-17. (canceled)

18. A method for producing a hypotensive agent containing an oligosaccharide that comprises at least one type of hexose and has at least one 1-6 bond, which comprises extracting a leguminous plant with water or an organic solvent.

19. The method for producing the hypotensive agent of claim 18, which comprises extracting a leguminous plant with water or an organic solvent, and then concentrating the resulting extract.

20. The method for producing the hypotensive agent of claim 18, wherein the leguminous plant is a black soybean.

21. A method of treating a patient to reduce hypertension, comprising administering to said patient in need of said treatment, a hypotensive agent comprising an oligosaccharide having at least one hexose and at least one 1-6 bond.

22. The method of claim 21, wherein the hexose is one ore more selected from the group consisting of glucose, galactose, mannose, altrose, talose, allose, idose, gulose, fructose, psicose, tagatose, sorbose, mannitol, altritol, talitol, iditol, galactitol, allitol, gulitol and glucitol.

23. The method of claim 21, wherein the hexose is one or more selected from the group consisting of glucose, galactose, mannose, fructose, mannitol and gulitol.

24. The method of claim 21, wherein the hexose is one or more selected from the group consisting of glucose, galactcose, mannose, and fructose.

25. The method of claim 21, wherein the oligosaccharide contains a constitutive unit of Glc(α1-6)Glc, Glc(β1-6)Glc, Gal(α1-6)Glc, Gal(β1-6)Gal, Glc(α1-6)Glucitol, Glc(α1-6)Mannitol or Glc (α1-6)Fru.

26. The method of claim 25, wherein the oligosaccharide contains a constitutive unit of Gal(α1-6)Glc, Gal(β1-6)Gal, Glc(α1-6)Glucitol, Glc(α1-6)Mannitol or Glc(α1-6)Fru.

27. The method of claim 26, wherein the oligosaccharide contains a constitutive unit of Gal(β1-6)Gal, Glc(α1-6)Glucitol, Glc(α1-6)Mannitol or Glc(α1-6)Fru.

28. The method of claim 21, wherein the oligosaccharide consists of glucose and/or galactose and has at least one 1-6 bond.

29. The method of claim 21, wherein all hexoses of the oligosaccharide bond by 1-6 bond.

30. The method of claim 21, wherein the oligosaccharide is a biose or a triose.

31. The method of claim 21, wherein the oligosaccharide is isomaltose, gentiobiose, melibiose, Gal(β1-6)Gal, glucitol, mannitol or palatinose.

32. The method of claim 31, wherein the oligosaccharide is melibiose, Gal(β1-6)Gal, glucitol, mannitol or palatinose.

33. The method of claim 32, wherein the oligosaccharide is glucitol, mannitol or palatinose.

34. The method of claim 21, wherein the oligosaccharide is derived from leguminous plants.

35. The method claim 34, wherein the oligosaccharide is derived from black soybeans.

36. The method of claim 21, wherein the oligosaccharide does not contain constitutive units of Glc(α1-3)Fru and Glc(α1-2B)Fru.

37. The method of claim 21, wherein the hexose is not raffinose.
